(19) United States
(12) Patent Application Publication
(10) Pub. No.: US 2015/0080751 A1
(43) Pub. Date: Mar. 19, 2015

(54) BLOOD PRESSURE MEASURING DEVICE, FLEXIBLE COLLAR FOR A BLOOD PRESSURE MEASURING DEVICE, AND METHOD FOR BLOOD PRESSURE MEASUREMENT

(71) Applicant: UP-MED GmbH, Munich (DE)

(72) Inventors: Stephan Regh, Munich (DE); Tobias Thomamueller, Bruckmuehl (DE); Reinhold Knoll, Neuburg (DE); Ulrich Pfeiffer, Munich (DE)

(21) Appl. No.: 14/376,206

(22) PCT Filed: Feb. 3, 2012
(86) PCT No.: PCT/EP2012/000500

§ 371 Date: Dec. 3, 2014

(51) Int. Cl. A61B 5/022; A61B 5/0225

(52) U.S. Cl. CPC A61B 5/02233; A61B 5/0225; A61B 5/02225
USPC 600/494; 600/499

(57) ABSTRACT

Blood pressure measuring device with a flexible pressure collar, to surround a body part at least partially, with a pressure sensor element, wherein the shape of the pressure collar can be adapted to the outer contour of the body part, the pressure collar being at least partially inelastic. Further, the flexible pressure collar for a blood pressure measuring device to the relates pressure collar being inelastic and the shape being adapted to the outer contour of a body part. A method for non-invasive blood pressure measurement on a patient's body part with a blood pressure measuring device includes: positioning the pressure collar on the body part; setting the pressure collar such that it exerts pressure in the pulsatile region of the patient on the body part; and recording the pulsation for the period of at least one breathing cycle of the patient in the form of pulsation signals.

The invention relates to a blood pressure measuring device, a flexible collar for a blood pressure measuring device, and method for noninvasive blood pressure measurement.

In many medically important situations, it is desirable to obtain information about the condition of the circulatory system of a human or animal patient. Particularly in the case of patients who require intensive medical treatment and care, medical staff are regularly required to take action to ensure the cardiovascular system responds in a specific way. Such an influence may be for example filling the circulatory system. However, one of the many critical factors to be considered when making such an intervention is the quantity of fluid that the patient needs and is able to tolerate. In this context, even the administration of various drugs that affect the cardiovascular system is to be considered an extremely delicate medical operation. In order to be able to reach a decision regarding the specific treatment strategy to follow, measurements have to be taken with the utmost reliability.

In this context, besides the blood pressure itself, the “heart-lung interaction” (HLI) is of primary importance. Mechanical ventilation of a patient who is to be treated causes the pressure in the chest to rise and fall, and these fluctuations in turn affect the filling of both the left and right sides of the heart. Consequently, the left ventricular stroke volume varies, and this in turn is reflected as a variation on the arterial blood pressure chart. In this context, typical HLI parameters include the stroke volume variation (SVV), the pulse pressure variation (PPV) and the pre-ejection phase variation (PEPV). However one drawback associated with these indices is that that they must be measured extremely accurately by invasively measuring arterial blood pressure. This requires cannulization/catheterization of an arterial vessel, which is time consuming, expensive and involves a high level of risk.

Various technical procedures are known in the field of non-invasive blood pressure measurement, which is considerably safer for the patient. For example, conventional procedures for measuring blood pressure are based on the capture of an acoustic signal in which the “Korotkoff sounds” are observed, but this is often susceptible to interference. In order to address this situation, U.S. Pat. No. 5,255,686 A1 for example provides a device and method for oscillometric blood pressure measurement, in which the capture of the interference-vulnerable acoustic signal containing of the Korotkoff sounds is replaced with a technically simpler pressure measurement system. The device provided for this consists of an air-filled collar with a sensor and a device for monitoring collar pressure and the temperature.

The principle of oscillometric blood pressure measurement is based on the observation that the pulse pressure wave created by the blood flowing through the artery causes the artery to dilate slightly, consequently causing a change in the shape of the collar. In a collar filled with air or fluid, this change in volume is converted into a change in pressure in accordance with the Gas Law. In this context, the volume changes in the artery are transformed into a change of shape of the collar. The forces that are necessary for this shape change result in a change of pressure in the fluid of the collar. This change of shape also takes place in the blood pressure measurement device that is suggested in DE 10 2009 039 257, for example. Thus, the collar pressure in such blood pressure measurement devices oscillates within narrow limits in response to the pulse pressure wave. The pulse pressure wave to be measured in the artery is also referred to as the pulsatile signal. It should also be noted that the oscillation amplitude of the signal is not constant. If a collar pressure slightly higher than systolic pressure is applied and then slowly reduced, it will be observed that the oscillation amplitude first increases until it reaches a maximum oscillation amplitude, and then diminishes again. It is assumed that the maximum oscillation amplitude occurs when the collar pressure is equal to the average blood pressure. Here, the average blood pressure is defined as the average over time of the arterial blood pressure during one heart beat. In this case, when the pressure of an air-filled collar equivalent to the systolic pressure is applied, the amplitude is approximately 45-57% of the maximum amplitude pressure, and when a collar pressure equal to the diastolic pressure is applied, the amplitude is approximately 75-85% of the maximum amplitude.

WO 2009/100927 A1 uses a pneumatic or hydraulic pressure collar, which serves to first determine the clamping pressure at which the maximum amplitude is reached in the oscillation process, and then, when this clamping pressure is applied, to measure the pulsatile fluctuations in the patient's arterial blood pressure with maximum amplitude. The drawback with this method, however, is that the gas- or fluid-filled collar has a damping and possibly even distorting effect on the pulsatile fluctuations that are being monitored. This can considerably compromise the signal quality and thus also the reliability of the method compared with invasive measuring methods. Consequently, reliable collection of HLI parameters with such a blood pressure measuring device is only possible to a very limited degree, if at all, without additional invasive measures, and involves significant additional effort.

It is therefore the object of the present invention to overcome these and other problems associated with the prior art and to provide an improved blood pressure measuring device. In particular it is intended to provide a blood pressure measuring device with which the signal quality of pulsatile signals in non-invasive measurement may be improved with respect to the prior art. It is a further object of the invention to provide an improved, flexible collar designed for use with a blood pressure measuring device according to the invention. Moreover, a method is to be provided for non-invasive blood pressure measurement that improves on the prior art. In particular the method is intended to enable improved capture of dynamic heart-lung interaction parameters (HLI) by non-invasive means.

This object is solved with the accompanying independent claims. Advantageous refinements are defined in the dependent claims.

In particular, the object is solved with a blood pressure measuring device having a flexible pressure collar that is configured to at least partially surround a patient's body part, and having a pressure sensor element, wherein the pressure collar is constructed as a mechanical pressure collar, particularly without fluid, preferably without a gas cushion, a gas mixture cushion and/or a fluid cushion, wherein at least part of the pressure collar is constructed inelastically, preferably unidirectionally inelastically, particularly preferably circumferentially inelastically, and wherein the pressure collar comprises at least one regulating device for mechanical regulation of the inner circumference of the pressure collar when it is attached to the body part. The circumferential direction of the pressure collar thus corresponds to the circumferential direction of the body part around which it is applied.

For the purposes of the present invention, the term “flexible” is understood to mean “pliable” or “adaptable in shape”, and the term does not refer to the expansion qualities of a material but only to the property of being able to assume various three dimensional shapes without suffering material breakage.

For the purposes of the invention, the term “mechanical” is understood to mean “non-pneumatic and non-hydraulic”. Similarly, the term “fluid-free” is understood to mean “free from fluids that are pneumatically or hydraulically usable or effective”. The mechanical, fluid-free design of the pressure collar refers exclusively to the pressure collar itself, and is not an essential property for the purpose of the invention, neither with regard to the design of the pressure sensor element nor for the design of the regulating device. To this extent, a blood pressure measuring device according to the invention may certainly include components that contain fluids, for example a gel pad in the area of the pressure sensor element, or a pneumatically or hydraulically controlled dynamic element in the area of the regulating device, the essential requirement being simply that the pressure collar that is passed around the body part to be measured exerts a force thereon by mechanical means.

The term “inelastic” is understood to mean “made from a stretch resistant material”. A “unidirectionally inelastic” material has a first direction, in which it is stretch resistant, and a second direction, in which it is stretchable—at least to a certain degree. Such a material may be, for example a textile fabric or textile-like fabric that is non-expanding and thus stretch resistant in the lengthwise direction but expandable in the transverse direction by virtue of the structure and the materials selected for the warp and weft thereof. In this context, the warp is usually understood to refer to a thread that extends in the lengthwise direction of the fabric and the weft is usually understood to refer to a thread that extends in the transverse direction of the fabric. For the purposes of the present invention, it is particularly advantageous if such a unidirectionally inelastic fabric is incorporated in the pressure collar in such manner that the inelastic direction of the fabric extends in the circumferential direction of the pressure collar. When such a pressure collar is passed around a body part it is able to adjust to the shape of the body part transversely to the circumference, but in the circumferential direction it is not elastic, which means that the encircled body part cannot expand beyond the inner circumference of the pressure collar.

A particular example of such a blood pressure measuring device according to the invention consists primarily in that the force exerted on the pressure collar due to the change in volume of the artery can be transmitted to the pressure sensor element almost entirely without damping. This is achieved in particular due to the fluidless and essentially inelastic construction of the pressure collar.

Consequently, a pressure collar according to the invention cannot expand either due to inadvertent tightening or twitching of the clamped muscles or by the arterial pulse pressure wave. To this extent the force exerted by the changing volume of the artery cannot be absorbed by the pressure collar but only by the pressure sensor element of the blood pressure measuring device according to the invention. In other words, all of the pressure exerted on the pressure collar by the arterial pulse pressure wave is absorbed by the pressure sensor element, which is preferably arranged between the body part to be measured and the pressure collar, and can be processed as a pressure signal almost without interference. At this point, the number of factors that are still to be considered mainly as capable of influencing signal quality is then determined only by the tissue composition of the body part which is to be measured and is encircled by the pressure collar, but not by the blood pressure measuring device itself. Such features may be, for example, the diameter of the body part and the proportions of fat, muscle and connective tissue contained therein. However, these factors can be captured and calculated out of the signal to be measured with the aid of a suitable algorithm. The pulsatile signal that is measurable with the blood pressure measuring device according to the invention then has a significantly improved signal-to-noise ratio. The oscillation amplitudes to be monitored can be measured in high signal quality and with low distortion.

It is easily deduced that the counterpressure the pressure collar must exert on the measured artery so that such a signal can be captured depends on the size of the inner circumference of the pressure collar relative to the outer circumference of the body part. Accordingly, a further advantage of the invention consists in that the inner circumference of the pressure collar is mechanically adjustable, with the aid of a regulating device according to the invention. As was noted previously, a mechanical regulation is understood to refer to a regulation process that is performed non-pneumatically and non-hydraulically. Such a regulation may for example consist in a shortening of the circumference of the pressure collar, caused by mechanically contracting the pressure collar. In this way, the pressure that is exerted on the body part by the pressure collar may be increased. A corresponding reduction of the exerted pressure may be brought about by loosening—enlarging the inner circumference—of the pressure collar.

A mechanical regulating device may have various forms. For example, it is conceivable that it is a regulating device that functions according to the iris diaphragm principle. In this case, the pressure collar may consist for example of a plurality of partial surfaces that are displaceable toward each other in such manner that the inner circumference of the pressure collar is adjustable.

In an advantageous embodiment of the invention, it is provided that the regulating device comprises at least one mounting device, at least one dynamic element and/or at least one force transmitting device. The regulating device is preferably a gear arrangement that has all three of said elements, that is to say a mounting device, a dynamic element and a force transmitting device. However, other variants that only embody one or two of said three elements are also conceivable.

A mounting device may be a stable or flexible plate or a fabric reinforcement. If present, the dynamic element and/or the force transmitting device may be mounted on or attached to the mounting device.

The dynamic element is preferably an element that provides a force, which is transmitted to the pressure collar by the force transmitting device and causes the pressure collar to loosen or tighten, and is thus able to cause the inner circumference of the pressure collar to become smaller or larger. In a preferred embodiment, the dynamic element is a motor.

The force transmitting device is preferably an element that directs the force exerted on it such that the inner circumference of the pressure collar is reduced or enlarged. For example, it is conceivable that the force transmitting device includes a drawstring or circular strap that is routed over one or more guide elements.

In a first, particularly easily realizable variant of the regulating device, the force may be exerted by a simple manual action on the force transmitting device. Thus, it is conceivable that the mounting device consists of holes with a reinforced border, which are created at opposite ends of a fabric strip that forms the pressure collar. The transmitting element may then be a string or string-like element that is threaded through the holes, and the ends of which are pulled to cinch the pressure collar together, in the manner of a corset. The holes thus function not only as a mounting device but also as guide elements for the force transmitting device. Of course it is also conceivable that deflection rollers, hooks, eyes or the like may be used instead of holes.

In a preferred variation, the regulating device comprises a motor as the dynamic element. Said motor may be mounted on the mounting device, for example. This may be a support plate, for example, the shape of which may more or less exactly match the contour of the body part.

In a first variant, a regulating device that is formed in this way may be constructed on the side of the pressure collar farthest from the body part, that is to say on the outside of the pressure collar. In an alternative variant, the regulating device may also be constructed on the inside of the pressure collar, that is to say on between the pressure collar and the body part where the measurement is to be taken.

In other design variants that may be combined with the embodiments described in the preceding, the regulating device may exert a force on the pressure collar from several different directions, thereby causing the change in the effective inner circumference of the pressure collar. The effective inner circumference of the pressure collar thus corresponds to the inner width of the blood pressure measuring device that is applied to the body part. It is subject to change due not only to the force exerted on the pressure collar but also the arrangement of the components that make up the blood pressure measuring device according to the invention.

The inner clearance and therewith the effective inner circumference of the pressure collar is essentially determined by the pressure collar itself and —if present—by the mounting device of the regulating device. In this context, the word “essentially” means that, for example, any gaps between the ends of the pressure collar that may be spanned by the regulating device, may contribute to the effective inner circumference of the pressure collar. However, no other components are used to form the inner circumference.

In a first embodiment it is provided that the pressure collar may be exposed to a force mechanically by the regulating device in the circumferential direction of the pressure collar. This is particularly helpful when the regulating device is positioned on the outside of the pressure collar. Accordingly, it is conceivable for example, that the regulating device comprises a drawstring or an element similar to a drawstring as a force transmitting device, the effective length of which—like the lacing and body shape adaptation of a corset—may be shortened or lengthened with the aid of said dynamic element, for example, if the dynamic element is able to wind up some or all of the force transmitting device. The pressure collar in this case may have the form of a band with two ends which may be draw toward each other as the force transmitting device is wound up, thereby reducing the inner circumference of the pressure collar.

In an alternative variant it is provided that the pressure collar may be exposed to a mechanical force from the regulating device in a direction radial to the axial direction of the body part. This may be advantageous, for example, if the regulating device is arranged inside the pressure collar. Then, for example, the pressure collar may be in the form of a closed ring. The effective inner circumference of the pressure collar is then determined by the pressure collar on the one hand and by the regulating device on the other. The regulating device may then be constructed in such manner that it presses the pressure collar outwards, that is to say radially to the axial direction of the body part encircled by the pressure collar. In this way the amount of the pressure collar that constitutes the effective inner circumference and consequently the entire effective inner circumference is reduced.

In both cases, the dynamic element may be a motor with a rotatable shaft. A coupling element may be present between the rotating shaft and the force transmitting device. For example, it is conceivable that the coupling element is a bobbin, a tappet, a cam or a component that is able to form a coupling.

In a first variant, this shaft may cause a change in the effective length of an element of the force transmitting device. For example, it may be coupled to a bobbin or bobbin-like element, wherein in the following the “bobbin” always refers to a bobbin-like element as well and includes the same. In this context the bobbin may be considered to be a coupling element. This is particularly beneficial if the regulating device is arranged on the outside of the pressure collar or if the force transmitting device is or comprises a thread-like element, which may be shortened or lengthened by winding or unwinding from the bobbin. Shortening, that is to say winding onto the bobbin, places tension on the thread-like element. One end of such a thread-like element of a force transmitting device may be attached to the pressure collar, while the other end may be attached to the bobbin or to the shaft of the motor, i.e., the dynamic element, which functions as the bobbin. Then, the pressure collar may be in the form of a strap, the two ends of which are facing one another when the pressure collar is placed around the body part to be measured, as described in the proceeding. The thread-like force transmission element may then connect the two ends to one another in a zigzag pattern, for example. The guide elements for such a force transmitting device may be deflection elements configured on both ends thereof, in the form of simple holes, or also rollers, hooks, eyelets, or similar, and through which the thread-like transmission element is threaded. Shortening the transmission element by winding onto the bobbin, that is to say onto the coupling element driven by the shaft of the dynamic element, then has the effect of drawing the two ends of the pressure collar toward one another and so reducing the inner circumference of the pressure collar. An observer would recognize that the improvement obtained as a result of the coupling element changing the effective length of an element of the force transmitting device, preferably a thread-like element of the force transmitting device, is evident particularly—but not only—when the regulating device exerts a circumferential force on the pressure collar.

In a further variant, the coupling element may cause an eccentric deflection of the pressure collar. The shaft of the dynamic element that is constructed as a motor may then be used as a driving means for a cam. Such a cam, which in this case functions as the coupling element, is preferably mounted on the shaft axis in such manner that it is rotatable about the shaft axis. It is then particularly advantageous if the regulating device is arranged on the inside of the pressure collar. Thus, it is conceivable, for example, that the dynamic element, that is to say the motor, is mounted on a support plate. The cam may then have one end that is distal and one end that is proximal to the axis, for example. The pressure collar, which is preferably in the form of a closed annular band, may then be guided via the cam. In such a case, the distal end of the cam, i.e., of the coupling element, is located closer to and farther from the support plate by turns as the cam rotates about the shaft axis. If the distal end of the cam also functions as a guide element for the pressure collar, the pressure collar will be deflected outwardly by the cam when the distal end thereof is located farther from the support plate. Consequently, in such a situation the inelasticity of the pressure collar causes the fraction of the effective inner circumference made up by the pressure collar is reduced relative to the support plate of the regulating device. This results in an overall reduction of the effective inner circumference. It will be noted that such a regulating device equipped with a cam exerts a force on the pressure collar in a direction radial to the axial direction of the body part the pressure collar surrounds.

In another variant it is conceivable that the regulating device causes a deflection of the thread-like element of the force transmitting device in a direction radial to the axial direction of the body part. Thus, for example, it is again conceivable that the pressure collar is a band, the short ends of which face each other when the band is placed around the body part. In this arrangement, the ends may slide at least over a section of the support plate of the regulating device. One end of the thread-like element of the force transmitting device may then be located at a certain distance from the first end of the pressure collar, and the second end of the device may be secured to the pressure collar at a certain distance from the second end of the pressure collar. Then, the dynamic element may be constructed in such manner that it drives a plunger, for example of pneumatic, hydraulic, or simply mechanical design, which in this case serves as the coupling element, so that the middle part of the thread-like element is forced outwardly thereby, radially to the axial direction of the body part. The two ends of the thread-like element are drawn together in this case and consequently cause the two ends of the pressure collar to move closer to one another as well. In this way the effective inner circumference of the pressure collar may be reduced further still.

It will be noted in each of the cases described and in each of the exemplary variants that it is advantageous for the purposes of the invention if a blood pressure measuring device according to the invention is equipped with a flexible pressure collar designed so as to at least partly encircle a body part, and with a pressure sensor element configured such that the pressure collar is designed as a mechanical pressure collar, particularly with no fluids preferably no gas cushion, no gas mixture cushion, and/or no fluid cushion, that the pressure collar is at least partly inelastic, preferably unidirectionally inelastic, particularly preferably circumferentially inelastic, and that the pressure collar has at least one regulating device designed to mechanically regulate the inner circumference of the pressure collar when it is placed around a body part. It will also be noted that in some of the cases and embodiments it may be advantageous for the regulating device to have at least one mounting device, at least one dynamic element and/or at least one force transmitting device, and if the force transmitting device has a tension element with adjustable position and/or length. In this case, the dynamic element may be connected to the force transmitting device either directly or with the aid of a coupling element.

In this case, it may also be beneficial of the regulating device comprises at least one cam as the coupling element. At any rater, it is advantageous if it is possible to apply a force to the pressure collar, via the regulating device, either mechanically in the circumferential direction of the pressure collar or mechanically in the in a direction radial to the axial direction of the body part.

It will also be noted that there are potentially many advantages to be gained if the regulating device of the pressure collar is equipped with a detent mechanism. Such a detent mechanism may consist for example in immobilizing the bobbin when a defined pressure is exerted by the pressure collar. This is particularly practical if pulsitile fluctuations are to be measured at predefined pressures, for example, over a certain period of time, or for comparison purposes with values collected previously in another measuring cycle. Thus it is conceivable that fluctuations of the mean arterial pressure may be measured during the course of one or more breathing cycles by a patient. This may be realized, for example, with the use of a self-limiting gear motor.

It is also advisable for a safety circuit to be present that is able to provide protection against excessively strong and/or excessively long application of force to the body part. This is particularly important in order to prevent circulation being restricted for too long. For example, electromagnetically actuated couplings between the drive shaft or transmission shaft and the bobbin or between the coupling element, the dynamic element and/or the force transmitting device are possible. In this case both force locking couplings such as conical couplings or magnetic couplings and form locking couplings such as claw couplings are considerable.

According to a further preferred embodiment, regardless of the configuration of the regulating device and the direction in which the regulation device exerts force on the pressure collar, it is provided within the scope of the invention and advantageous if the pressure collar is conformable to the outer contour of the body part. This ensures that the contact pressure of the collar is distributed evenly. The flexible pressure collar of the blood pressure measuring device according to the invention is thus able to be used on extremities of all shapes, for example, with a cylindrical, conical or pear-shaped.

In a further preferred embodiment, the pressure collar consists of partial surfaces that are moveable relative to each other. It is thus conceivable, for example, that the pressure collar has the basic form of a rectangular strap, which can be placed around the body part. Of course, other basic shapes are conceivable, such as the shape of a lateral surface of a truncated cone or a closed annular band. The partial surfaces may be for example, essentially rectangular strips arranged and extending parallel to each other. The strip-like partial surfaces may be arranged side by side to form a rectangular base surface. In such case, preferably at least three such strips are present. It is conceivable that each of the strip-like partial surfaces has one long and one short side, wherein the long side of the partial surfaces either may be aligned parallel to the long side or parallel to the short side of the basic body. In such case, when it is applied, the long side of the basic body preferably extends circumferentially about the body part, while the short side of the basic body extends preferably axially, that is to say, parallel to the longitudinal axis of the body part. Accordingly, the long sides of the partial surfaces also extend preferably in the circumferential direction. Each of these partial surfaces may then be equipped, for example, with its own regulating device, for instance, a regulating device having a cam as described above. However, it is also conceivable that the partial surfaces are regulated by a common regulating device, for instance, if they are connected or coupled via a thread-like element of a force transmitting device.

The partial surfaces may also form a spiral strap, that is placed around the body part. In this context, it may be envisaged that the partial surfaces have a different shape, for example, the shape of triangles or other polygons.

The fact that the partial surfaces are able to move relative to each other allows them to lay in particularly form fitting manner of the surface of the body part to be measured. It is particularly favorable if the partial surfaces are made from a flexible but not expandable material. In a further preferred embodiment the partial surfaces are therefore preferably flexible, particularly flexible and at least partly inelastic. Hereto it is conceivable that the inelasticity of the partial surfaces is a unidirectional property of the material from which the partial surfaces are made. In this way, the individual partial surfaces are able to conform optimally to the surface area of the body part against which they are lying. However, they do not yield to a pressure exerted on them by a fluctuation in the arterial pressure of the body part.

It will also be noted that it is beneficial if the partial surfaces are connected with each other. The interconnected partial surfaces of the pressure collar are thus able to move relative to each other, similarly to a joint. This promotes the optimum conformation of the pressure collar to the outer contour of the body part. This enables the partial surfaces to cover the surface of the body part over the entire contact area of the pressure collar, without creating creases or sites of contactless air pockets. This in turn is beneficial for the even transfer of force from the pressure collar to the body part that is to be measured.

The interconnection of the partial surfaces is carried out preferably with the aid of connecting elements. In the simplest case, these may be strips of the same material from which the partial surfaces are made, arranged between the partial surfaces. Thus, it is conceivable for example, that the flexible pressure collar itself, as described previously, is constructed from a large rectangular strip of material. However, this material is preferably flexible but not expandable, that is to say, inelastic. Then, rows of slits may be created in these material strips, parallel and at a certain distance from each other. These rows of slits divide the rectangular base body of the flexible pressure collar into the partial surfaces according to the invention. The connecting elements can then be formed by remaining fillets of material arranged between the slits. The connecting elements are therefore also preferably flexible. In this case, a certain amount of axial elasticity in the connecting elements may be tolerated within certain limits. It is also possible that the pressure collar consists of a material strip made from a nonexpanding fabric. This material strip may be divided into partial surfaces by the selective removal of warp threads of the fabric. Subsequently, an isotropic, flexible connection of the partial surfaces may be created by the weft threads of the fabric at those sites where the warp threads were removed.

It is also conceivable for the connecting elements to be made from a different material than the flexible pressure collar. In general, the connecting elements perform the function of articularions that enable the partial surfaces to move relative to each other, as described in the preceding. In a further preferred embodiment, the invention provides that the pressure collar has connecting elements that preferably connect the movable partial surfaces to each other.

With regard to the use of a blood pressure measuring device according to the invention, it is conceivable for example to initially place the blood pressure measuring device together with the flexible pressure collar loosely around the body part to be measured. Then, the inner circumference of the loosely encircling pressure collar may be gradually constricted with the aid of the regulating device until the pressure collar is in full contact with the body part, and cannot slip or be dislodged. Then, the inner circumference of the pressure collar may be constricted further, tightening around the body part that is to be measured. As a result, an artery in the body part to be measured is constricted by the surrounding tissue structures. When the pressure exerted on the body part by the pressure collar is greater than blood pressure in the artery, the flow of blood in the artery is interrupted. It should be noted that the flow of blood is interrupted while the pressure is greater than the diastolic but less than the systolic pressure, in intervals that match the heart rhythm. As soon as the pressure exceeds the systolic pressure in the artery, blood flow stops altogether.

The pressure sensor element of the blood pressure measuring device according to the invention is preferably arranged on the side of the pressure collar facing the body part. Accordingly, when the blood pressure measuring device is applied to the body part, the final position of the pressure sensor element is between the body part and the pressure collar. It is then pressed against the body part by the pressure collar. Since, as explained in the preceding, the pressure collar preferably exerts pressure on the body part without the aid of fluid cushions, particularly without air cushions, since it is inelastic and also since—because of its ability to adapt to the contour of the body—the pressure it exerts is applied perfectly evenly over the entire contact surface, the conditions are created under which the pressure sensor element is able to measure only the pressure pulses transmitted from the body part. In this sense, it functions as a kind of counterbearing for the force exerted by the arterial pulse pressure wave, and prevents the pulse signals from being damped as they are transmitted to the pressure sensor or pressure sensor element.

The pressure sensor element preferably consists of a gel pad, in which the pressure sensor is embedded. The gel pad assures a flexible, full-contact, hydraulic coupling between the pressure sensor and the body part concerned.

In this respect, a preferred blood pressure measuring device is notable because it comprises a flexible, mechanical pressure collar, which is adaptable to the outer counter of the body part. The pressure collar preferably consists of partial surfaces that are movable relative to each other, flexible but inelastic, and connected to each other by connecting elements, and has a regulating device. The blood pressure measuring device further comprises a pressure sensor element that is preferably arranged between the body part for which the HLI and other hemodynamic parameters such as the stroke volume of the heart and the cardiac output are to be determined and the flexible pressure collar of the blood pressure measuring device. The blood pressure measuring device is preferably designed such that the flexible pressure collar is able to hold the pressure sensor element firmly in place opposite an artery that is to be measured without introducing any damping or distorting effect on the signal that is to be measured. In other words, the blood pressure measuring device designed for noninvasive, low noise, low distortion measurement of blood pressure, HLI, and hemodynamic parameters.

In another embodiment, it is also conceivable that the pressure sensor element of a blood pressure measuring device according to the invention is applied to the side of an additional collar that can be made more rigid and is also of purely mechanical design, facing towards the body. The flexible pressure collar is then not positioned in direct contact with the body part and the pressure sensor element but encircles said collar and is wrapped around the said second collar. In this case, the pressure is transmitted to the body part from the pressure collar via the tensionable collar.

In a further aspect the invention relates to a flexible pressure collar for a blood pressure measuring device according to the invention. Such a flexible pressure collar is notable for the fact that it is designed as a mechanical pressure collar, particularly without the use of fluids, preferably without the use of gas cushion, gas mixture cushion/or fluid cushion, that it is at least partially inelastic, preferably unidirectionally inelastic, particularly preferably inelastic in the circumferential direction thereof, and that in the applied condition it has at least one regulating device for the mechanical regulation of the inner circumference of the pressure collar. In this context, it is also particularly advantageous if the pressure collar consists of partial surfaces that are movable relative to each other. Furthermore, it is useful if the shape of the pressure collar can be adapted to match the outer contour of a body part. The pressure collar, in particular the regulating device of the pressure collar may have the form already described with reference to the blood pressure measuring device.

With such a flexible, mechanically constructed, and preferably inelastic pressure collar it is possible to achieve good conformation and even distribution of the contact pressure for any body part shape “cylindrical, conical, pear shaped or similar”. In a particularly simple embodiment, the flexible pressure collar consists of a material strip wound in a spiral about the body part. In this case, the individual spiral windings may represent partial surfaces of the pressure collar and may be connected to each other with the aid of connecting elements. In this context the spirals may comprise multiple partial surfaces. According to a preferred embodiment, the flexible pressure collar consists of partial surfaces that are movable relative to each other. As was explained above, each partial surface may be equipped with its own regulating device, or the pressure collar may have a regulating device that regulates all of the partial surfaces. Also as explained in the proceeding, it is conceivable, for example, that the pressure collar may consist of a rectangular base body that is divided into multiple, also rectangular strips extending parallel to each other, as described above. In this context, at least three or more strips are preferred. The partial surfaces at the edges of the flexible pressure collar may be offset with respect to the other partial surfaces. This enables good conformation with the corresponding body part.

In a further preferred embodiment, it is provided that the partial surfaces of the pressure collar are flexible and inelastic. This enables them to be bent into shape, and so follow the contour of the body part. At the same time, they do not yield to the pulsatile arterial pressure acting on the collar. Accordingly, no damping effect of the pulsatile arterial pressure signal to be measured is created by the pressure collar itself.

In a further preferred embodiment, it is provided that the pressure collar comprises connecting elements that preferably connect the partial surfaces to each other. As was described previously, these connecting elements function in the same way as joints between the partial surfaces. They are preferably fillets of the same material from which the partial surfaces are also produced. In a particularly simple embodiment, the flexible pressure collar then typically consists of a rectangular base body made from a corresponding, inelastic material in which rows of lengthwise, parallel slits are created. The rows of slits divide the rectangular base body into the partial surfaces. Fillets of the material remain between the rows of slits. These fillets then form the connecting elements between the partial surfaces.

In a further embodiment, the invention provides that the regulating device of the pressure collar is preferably constructed such that the forces it exerts are distributed equally among all partial surfaces of the flexible pressure collar. As was explained in the preceding, the pressure collar may have for example a rectangular base body with two long sides and two short sides. The two short sides may then be arranged opposite one another when the flexible pressure collar is placed around the body part that is to be measured, for example. The regulating device is then arranged for example on the short sides of the rectangular base body, and is than able to connect the two short sides to one another.

In this context, it will be noted that it is advantageous if the regulating device comprises at least one motor. It is also favorable if the regulating device comprises a force transmitting device, preferably a toothed belt or a drawstring. The force transmitting device may be shortened as described above, for example, with the aid of the motor, so that the flexible pressure collar is drawn together and exerts the corresponding force on the body part that is to be measured. At the same time, it is also conceivable that a plurality of motors is used. If this is the case, they are preferably connected in series in order to obtain proportional motor torques. The net result is that the flexible pressure collar may be shortened and pressed against the arm with the aid of the regulating device.

If the force transmitting device is a drawstring, it is conceivable, for example, that deflecting elements may be conformed on the ends of the various partial surfaces that make up the pressure collar, in particularly on the short sides of the pressure collar, and the drawstring may be supported on said deflecting elements. The deflecting points may be mounted rollers or slide bearings, for example, and may serve to guide the string of the drawstring.

The one end of the pressure collar may comprise a support element, in the form of a base plate, for example. This is preferably the mounting device for the regulating device. The motor and a first group of deflection elements may be mounted on the support element. The partial surfaces of the pressure collar that form the end of the pressure collar may be fastened to the support element by one end. As indicated in the preceding, the partial surfaces are preferably made from strips that are arranged longitudinally parallel to each other. If the strips are rectangular strips of which the long sides correspond to the long side of the base body of the pressure collar, when the pressure collar is arranged in a ring the ends of the partial surfaces that are not attached to the base plate are positioned opposite each other. One or more support elements may also be conformed on these ends of the partial surfaces. These are useful particularly to ensure stability when a second group of deflection elements is mounted. For example, they can prevent the deflection elements from being ripped out of the partial surfaces of the pressure collar instead of helping to draw the pressure collar together as soon as a corresponding tensile force is applied to them.

In a preferred embodiment, the deflection elements, deflection rollers, for example, are arranged on both ends of the pressure collar, for example on the support elements created there. The force transmitting device, in the form of a drawstring, for example, may then be routed around the deflection elements in such manner that the two ends of the pressure collar joined with each other in a zigzag pattern by the force transmitting device of the regulating device. The one end of the force transmitting device, which preferably has the form of a drawstring, may thus be secured to the support element that is in the form of the base plate. From there, it is threaded around the individual deflection elements, which are preferably in the form of rollers, particularly in alternating manner around a deflection element located on the support element in the form of a base plate, and then around a deflection element arranged on the opposite end of the pressure collar. The other end of the force transmitting device, in the form of a drawstring or a v-belt, may be wound onto a coupling element, for example a bobbin, which is connected to the motor. In this case, when the motor causes the force transmitting device to become shorter, for example by winding the drawstring onto the bobbin, the force transmitting device pulls the two ends of the pressure collar toward one another, thereby also reducing the circumference of the encircled body part. In this way, the individual partial surfaces of the pressure collar are drawn toward the outer circumference of the body part, and the same force is transmitted to each partial surface. In this way, the pressure collar conforms to an enormously wide range of limb geometries and is pressed evenly against the body part. If the drawstring is then unwound from the bobbin again, the tissue pressure from the previously constricted body part forces the flexible pressure collar apart again. The pressure may be relieved evenly.

A preferred flexible pressure collar for a blood pressure measuring device according to the invention is notable in one respect in that it consists of an inelastic, that is to say non-expanding, but flexible material. It has a base body that consists of a number of partial surfaces, and which are connected to each other via connecting elements, and are also made from said inelastic but flexible material. The pressure collar further comprises a regulating device, which consists of a force transmitting device and a motor. The regulating device preferably comprises a plurality of deflection elements, of which a first group is arranged on the first end of the base body of the pressure collar, and a second group is arranged on the second end of the base body of the pressure collar. In this arrangement, the deflection elements, the force transmitting device and the motor are mounted on the top or side of the support elements. It is also conceivable for the deflection elements to be embedded in the one or more support element(s), or they may be at least partly embedded therein. Particularly if the deflection elements are rollers and the force transmitting device is a drawstring, this serves to prevent the drawstring from slipping off the rollers due to accidental external influences.

In a further aspect, the invention relates to a method for non-invasively measuring blood pressure in a body part of a patient using a blood pressure measuring device according to the invention, which method comprises the following steps:
a) placing the flexible pressure collar around the body part;
b) adjusting the flexible pressure so that it exerts a pressure in the pulsatile range on the body part;
c) recording the pulsation in the form of pulsatile signals for the duration of at least one breathing cycle of the patient.

At the same time, the pressure collar is used to position the pressure sensor element on the body part. The pressure sensor element is attached to the pressure collar before it is placed around the body part, so a separate step does not need to be carried out to place the pressure sensor element on the body part. However, of course it is also possible to attach the pressure sensor element to the body part first, and place the pressure collar on top of it.

The pressure collar is adjusted in such manner that the pressure collar exerts either a constant or a fluctuating pressure. This is set as required, particularly depending on the desired parameters the operator wishes to collect.

With such a method, it is possible to reliably measure fluctuations in the arterial blood pressure chart, which may be used as an index for heart-lung interaction, the heart stroke volume and the cardiac output, by means of a pulse contour method. It is also particularly advantageous of the method is performed using a blood pressure measuring device according to the invention, particularly using a flexible pressure collar according to the invention. The method is based on the recording of pressure curves that are in proportion with the arterial pressure curves. Such a method is obviously not a therapeutic procedure, since the patient receives not therapeutic treatment. It is also not a diagnostic procedure, since the measured parameters per se do not enable diagnosis of a specific disease, but rather determine selective parameters that require more detailed interpretation, or have to be combined with other information or data to permit a diagnosis.

In a preferred embodiment, the adjustment of the pressure collar in step b) comprises the steps of
i) exerting a continuously rising pressure on the body part via the pressure collar, until the pressure collar exerts a pressure in the pulsatile range of the patient;
ii) measuring the amplitude of the pulsations;
iii) continuously increasing the pressure until the amplitudes of the pulsations fall back to a predetermined fraction of the measured maximum amplitude;
iv) loosening the pressure collar until a previously calculated pressure value is reached, for example between diastolic and systolic pressure;
v) locking the pressure collar at the pressure value set in step iv).

In this method, the procedure steps are not carried out sequentially, but largely concurrently or cyclically. In particular, the amplitude maximum is determined continuously.

The recording of the pulsation in step c) is carried out in this manner with a collar pressure having the best signal quality, that is to say with a collar pressure for which a signal with sufficient amplitude and with very low distortion reaches the collar. Fluctuations in pulse pressure and the pressure curve shape that are attributable to the heart-lung interaction may thus be captured with a very high degree of accuracy.

In a further preferred embodiment, it is provided that the pulsation signals recorded in step c) may be linearized preferably with the aid of a model calculation, particularly preferably with a model calculation containing sigmoidal transfer elements.

Other features and properties of the invention will be evident from the following description of special embodiments and with reference to the drawing. In the drawing:

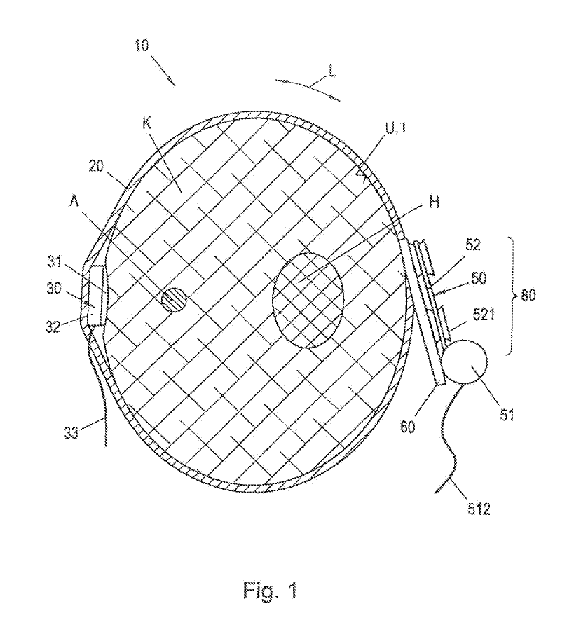

FIG. 1 is a schematic cross section through a body part with a pressure measuring device according to the invention applied thereto;

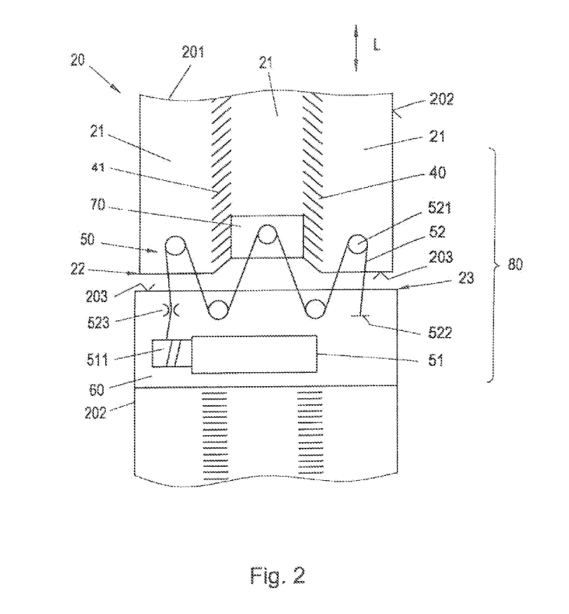

FIG. 2 is a detail view of the connecting area of a flexible pressure collar according to the invention;

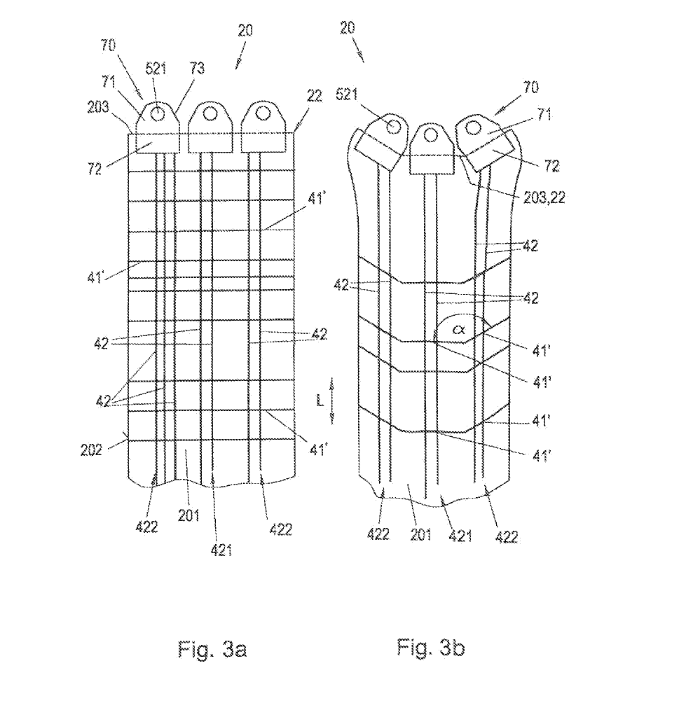

FIG. 3a is a schematic representation of a [front end] of a pressure collar according to the invention;

FIG. 3b is a schematic representation of the pressure collar of FIG. 3a in a condition in which it is applied to a biceps-shaped body part;

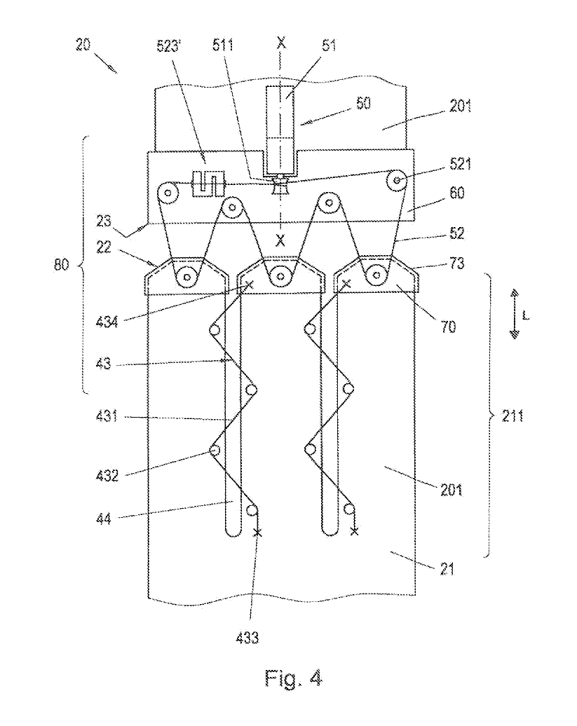

FIG. 4 is a detail view of a connecting area of another embodiment of a flexible pressure collar according to the invention;

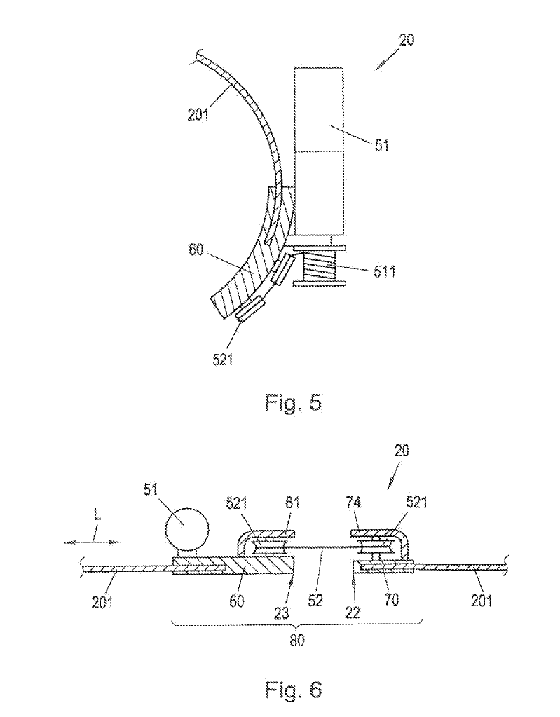

FIG. 5 is a cross section through the connecting area of the flexible pressure collar, of FIG. 4 along line X-X;

FIG. 6 is a side view of a connecting area if a flexible pressure collar according to the invention;

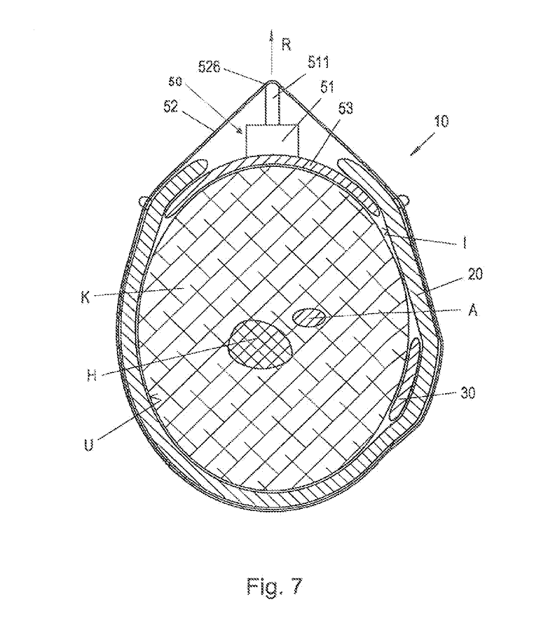

FIG. 7 is a schematic cross section through another variation of the blood pressure measuring device according to the invention applied to a body part;

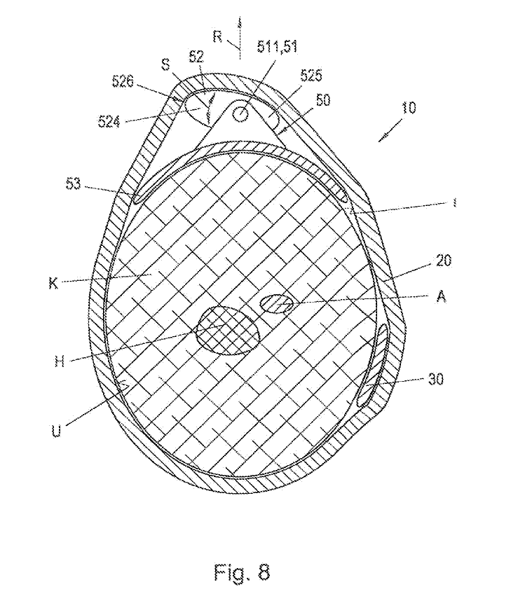

FIG. 8 is a schematic cross section through a further variation of the blood pressure measuring device according to the invention applied to a body part;

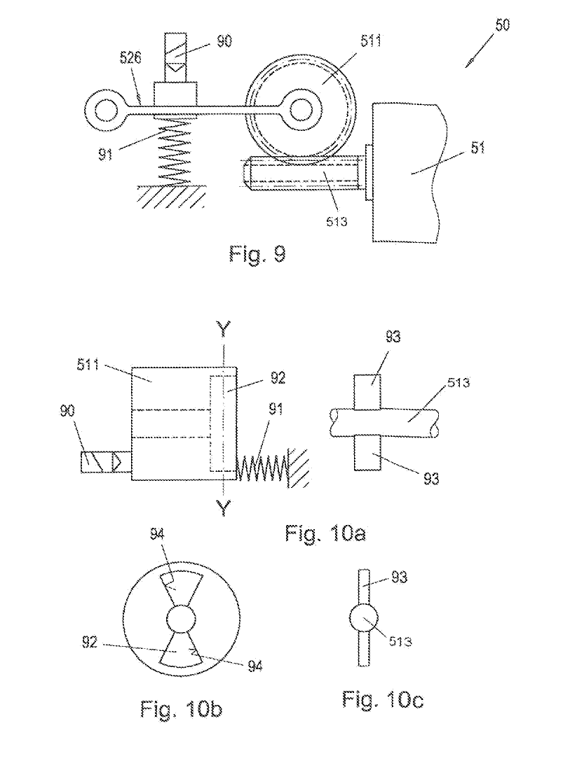

FIG. 9 is a schematic representation of a safety circuit for a mechanical regulating device according to the invention;

FIG. 10a shows a further embodiment of a safety circuit for a mechanical regulating device according to the invention;

FIG. 10b is a cross section through the safety circuit of FIG. 10a along line Y-Y;

FIG. 10c is a detail view of a shaft of a dynamic element of a safety circuit of the embodiments shown in FIG. 10a.

FIG. 1 shows a cross section through a body part K, here an arm, in which a bone H, here a bone of the upper arm (Os humeri), and an artery A, here the brachial artery {Arteria brachialis). A blood pressure measuring device 10 according to the invention is placed around the outer circumference U of body part K. Blood pressure measuring device 10 consists of a pressure collar 20 and a pressure sensor element 30. Pressure collar 20 is made from an inelastic but flexible material, for example polyamide, polyester, polyethylene or polypropylene. Because of its flexible construction, the device can adapt itself to the external shape of body part K, particularly the contour along circumference U. At the same time, however, its inelastic construction means that it does not yield in response to an expansion of body part K—again, particularly an expansion of circumference U—as soon as the inner circumference has adapted to the shape of body part K. It will be noted that shape of pressure collar 20 can be adapted to fist the outer contour of body part K. Moreover, the body part K encircled by pressure collar 20 is forced into a stress-optimized cross section by the nature of the pressure exerted on it. This stress-optimized state then does not change its shape if the tissue pressure is increased.

Pressure collar 20 encircles body part K in the lengthwise direction L and along the entire circumference U thereof. A connecting area 80 is shown in FIGS. 1 and 2, and this is where a first and a second end 22, 23 (see also FIGS. 2, 3A, 3B, 4, 6) of pressure collar 20 are connected to each other. A regulating device 50 is constructed on said connecting area 80, and may be used to shorten the length of pressure collar 20 in such manner that artery A is constricted by the increasing tissue pressure. As a consequence, the flow of blood through artery A is reduced or completely stopped.

Regulating device 50 comprises a force transmitting device 52. Said force transmitting device 52 is exposed to a force by means of a dynamic element 51, which is a motor, which force can cause the inner circumference of pressure collar 20 to be constricted or expanded. In the embodiment shown, force transmitting device 52 is a drawstring which is routed variously with the aid of a plurality of deflection elements 521. At the same time, the force exerted by regulating device 50 is distributed evenly to the first and second ends 22, 23 of pressure collar 20. It will be noted that regulating device 50, particularly the deflection elements 521 conformed on the first end 22 of pressure collar 20, and the dynamische element 51 in the form of a motor, are all mounted on a support element 60.

Constructed as a motor, the dynamic element 51 of regulating device 50 is connected to a power cable 512. Through power cable 512, the dynamic element 51 in the form of a motor may be supplied with the electrical energy it needs to operate, and control data that determine the order of the force that is exerted on force transmitting device 52 by regulating device 50.

Pressure sensor element 30 consists of a sensor 31 and a measuring device 32, which is connected to a power cable 33. In the example illustrated, sensor 31 is a gel pad in which a pressure sensor is embedded. In an alternative embodiment (not shown), sensor 31 may also be a fluid cushion, which is connected to a sensor arranged outside blood pressure measuring device 10 via a hose or similar connecting member, wherein the pressure fluctuations are transmitted from the fluid cushion to external sensor 31 according to the principle of communicating pipes.

As is shown in FIG. 1, after blood pressure measuring device 10 according to the invention is put in position, sensor 31 is positioned in such manner that it is lying against body part K, immediately opposite artery A. Pressure collar 20 presses pressure sensor element 30 against body part K such that it is unable to move in response to the pulsatile pressure curve signal that is emitted from body part K, and is thus able to record the pulsatile tissue pressure curves and the fluctuations thereof without any form of damping. It will be noted that an arrangement sequence from the outside inwards is formed. The outermost element is pressure collar 20, followed by pressure sensor element 30 or sensor 31, then by the tissue of body parts K, followed lastly by artery A, and again followed by tissue of body part K and bone H. The changes in pressure within artery A, which may be measured as pulsatile waves or fluctuations, may spread as far as pressure sensor element 30 in this way, without the damping influence of blood pressure measuring device 10. Because of the inelastic construction of pressure collar 20, pressure sensor element 30 is able to receive the pressure waves from the tissue practically unmodified. Any distortions that might arise due to the tissue of body part K may be detected and compensated for using a corresponding algorithm. This is made possible particularly by the fact that pressure collar 20 is always optimally positioned against corresponding body part K. This is achieved through the special design of pressure collar 20.

Accordingly, FIG. 2 shows that pressure collar 20 consists of an essentially rectangular base body 201. Said base body has two oppositely positioned long sides 202 and two short sides 203. In the applied position, short sides 203 face one another. In this situation, the one short side 203 may be conformed on first end 22 of base body 201, while the second short side 203 is conformed on the second end 23 of base body 201.

FIG. 2 also shows that base body 201 of pressure collar 20 consists of two partial surfaces 21, which extend in the lengthwise direction L of pressure collar 20. Partial surfaces 21 are connected to each other by means of connecting elements 40. In the present example of FIG. 2, both partial surfaces 21 and connecting elements 40 are formed by creating two rows of cutaways 41, which may also be referred to as slits, running parallel to each other in base body 201 of pressure collar 20. In the example shown, cutaways 41 run at a slight angle to the lengthwise direction L of pressure collar 20 because of the tensile stress caused by adapting to body part K. In the unstressed state, cutaways 41 are preferably aligned transversely to lengthwise direction L. However, it is entirely conceivable for the cutaways to be aligned parallel or at any angle with respect to lengthwise direction L. It will be noted that partial surfaces 21 are separated from each other by the rows of cutaways 41. Connecting elements 40 are fillets of the same material as the base body is made from, which remain between the individual cutaways 41, and thus connect partial surfaces 21 integrally to each other.

It will be further noted from FIG. 2 that first end 22 and second end 23 of the base body 201 of pressure collar 20 are connected to each other in connecting area 80 with the aid of regulating device 50. The connection is created using force transmitting device 52, in the present example a drawstring, which is wound round a plurality of deflection elements 521. Deflection elements 521 of the example shown are rollers, which are formed on both the first end 22 and the second end 23 of the pressure collar. Force transmitting device 52, in this case the string of the drawstring, is wound back and forth in a zigzag pattern between the roller-like deflection elements 521 of first end 22 and second end 23. In addition, the one end of force transmitting device 52 is wound round a coupling element 511, which is in the form of a bobbin, of a dynamic element 51 that has the form of a motor. When the dynamic element 51 that is realized as a motor is turned, force transmitting device 52 amy be made shorter or longer. In this configuration, it is practical for the other end of force transmitting device 52 to be attached to a fastening element 522. When force transmitting device 52 is shortened, it causes pressure collar 20 to contract, and when force transmitting device 22 is lengthened, the inner circumference of pressure collar 20 is enlarged. If the inner circumference of pressure collar 20 becomes smaller than the circumference U of body part K, pressure collar 20 exerts a corresponding pressure on body part K. It is further shown in FIG. 2, that dynamic element 51 of regulating device 50, which element is in the form of a motor, is mounted on a support device 60 that is conformed on the second end of pressure collar 20. Deflection elements 521 of the second end 23 of pressure collar 22 are also created on support device 60. There is also a support device 70 on the first end 22 of the pressure collar. Support devices 60, 70 are particularly helpful with regard to providing stability for pressure collar 20 in connecting area 80.

FIG. 2 also shows that a guide element 523 is present on support device 60. Guide element 523 guides force transmitting device 52 from coupling element 511 to the first deflection element 521, which is located on the facing, first end 22 of pressure collar 20. In an alternative embodiment, such as is shown in FIG. 4, a force sensor 523′ may be provided instead of the guide element 523, to monitor the force that is exerted on pressure collar 20 by force transmitting device 52. A combination solution of guide element 523 and force sensor 523′ is also possible.

FIGS. 3a and 3b show s further embodiment of the flexible pressure collar 20 according to the invention. In this, FIG. 3a shows pressure collar 20 in a condition not applied to a body part K, and FIG. 3b shows pressure collar 20 in a situation in which it has been applied, for example to a biceps-like body part K. This causes a bulge to appear in pressure collar 20 which is seen vertically from above in FIG. 3b, so that the bulge appears flat. However, the bulge of pressure collar 20 is not caused by an expansion of the material in lengthwise direction L, and it is essentially also not associated with such an expansion. Base body 201 of the pressure collar 20 shown in FIGS. 3a and 3b here consists of a fabric made from inelastic lengthwise threads 42 which are interwoven with transverse threads 41′ and in this way connected movably with each other. In this context, only a few examples of longitudinal 42 and transverse 41′ threads are shown in FIGS. 3a and 3b, simply for illustrative purposes in each case. The fabric is preferably of inelastic construction, at least in the lengthwise direction of pressure collar 20. Lengthwise threads 42 are aligned parallel to the lengthwise direction L of pressure collar 20, which corresponds to the circumferential direction, that is to say the direction of circumference U when the collar is in position on a body part. Lengthwise threads 42 may be considers partial surfaces under these circumstances. Lengthwise threads 42 may be shifted in the lengthwise direction parallel to each other, for example when pressure collar 20, as shown in FIG. 3b, is placed around a biceps-shaped body part. Particularly the outer lengthwise threads 422 may be shifted in lengthwise direction L relative to the inner lengthwise threads 421 without expanding in a lengthwise direction. Transverse threads 41′ are able to follow this shift, with the result that the transverse threads 41′ may be bent by an angle α. In such as case, transverse threads 41′ may possess a small degree of elasticity. In this contaxt, the flexibility of pressure collar 20 may be determined by the nature of the weaving of transverse and longitudinal threads 42, 41′. Accordingly, different veaving styles, such as Atlas or Köperbindung may be conceivable depending on the need. The use of different thread types can also be envisioned, as is the introduction of divisions according to whether one or more transverse threads 41′ at a time is/are left out at certain intervals.

The embodiment of FIGS. 3a and 3b also has three support devices 70 arranged on each short side 203 of first end 22, on which deflection elements 521 are provided. It will be noted that each support device consists of a first section 72 and a second section 71. First section 72 is connected to base body 201, while second section 71 supports deflection element 521. In the embodiments shown, border 73 of second section 71 is at an angle, so that support devices 70 can be moved relative to each other with obstructing the movement of the other. This is particularly advantageous when support devices 70 are arranged at an angle of α relative to each other when the collar has been placed around body part K.

FIG. 4 shows a further embodiment of the design of connecting section 80 of a flexible pressure collar 20 according to the invention for a blood pressure measuring device 10 according to the invention. Here too, the base body 201 of pressure collar 20 consists of a plurality of partial surfaces 21, which are movable relative to each other. Again, support device 60 is arranged on the second end 23 thereof, on which a dynamic element 51 in the form of a motor is arranged centrally with a coupling element 511 in the form of a bobbin that belongs to a regulating device 50. Regulating device 50 also includes a number of deflection element 521, which are diposed on support device 60 and on opposing support device 70 of the first end 22 of pressure collar 20. Besides this, a force sensor 523′ is arranged on support device 60 of the second end 23 of pressure collar 20, and measures the force that is being applied to the force transmitting device. The measured force may be used to determine how much pressure is being used to press the pressure collar 20 together, and correspondingly, what pressure is exerted by pressure collar 20 on the body part K (not shown in FIG. 4) that is encompassed by pressure collar 20.

FIG. 4 also shows that end area 211 of base body 201 is constructed on first end 22 such that the partial surfaces 21, which extend parallel to each other in lengthwise direction L of the base bodies 201, are separated from each other by cutaways 44. In this situation, however, the partial surfaces 21 are connected to each other by means of a joining device 43. The joining device 43 prevents the cutaways 44 from becoming so far apart when pressure collar 20 is used that the pressure measurement is affected thereby. Joining device 43 consists of a joining element 431, which may be a thread, for example. Joining element 431 is secured to an upper fastening point 434 and a lower fastening point 433 on each partial surface 21 of base body 201 of pressure collar 20. Each joining device 43 joins two adjacent partial surfaces 21. For this purpose, joining element 431 is guided back and forth in a zigzag pattern via cutaway 44. Deflection points 432 are provided on each partial surface 21 for this purpose. The deflection points 432 in the present example are deflection rollers, but they might equally well be eyelets, deflection pins, hooks or the like. It may be seen that the upper fastening point 434 is preferably formed in the area of support device 70. This allows a particularly stable fastening arrangement. Moreover, it may be noted that each partial surface 21 is furnished with its own support device 70. Here too, borders 73 of the support device are chamfered.

FIG. 5 shows a cross section through the pressure collar 20 according to the invention in the mounted position, along sectional line X-X in FIG. 4. Support device 60 may be rigid, but may have been bent before mounting, wherein base body 201 is flexible. This enables body part K and support device 60 as well as base body 201 to adapt perfectly to each other. At the same time, support device 60 and base body 201 are not elastic, which means that they cannot yield in response to a fluctuation in circumference U of body part K, for example if the muscles are tensed, or due to fluctuations in arterial pressure. The circumference of pressure collar 20 is regulated solely by regulating device 50. It will be noted that when the device is applied, dynamic element 51 in the form of a motor is arranged on support device 60 such that it does not inhibit a change or adaptation of the inner circumference of pressure collar 20 to the corresponding body part K. The same applies for deflection elements 521.

FIG. 6 also shows a cross section through an area of a flexible pressure collar 20 according to the invention. This shows schematically how the support devices 60, 70 are disposed on base body 201 and are facing each other in connecting area 80. Dynamic element 51 in the form of a motor is also positioned on support device 60. A deflection element 521 is visible in the cross section on each of support device 60 and support device 70. Force transmitting device 52—in the example shown, a drawstring—is threaded around deflection elements 521 and regulates the gap between second end 23 and first end 22 in connecting area 80. Whe force transmitting device 52 is shortened, the width of this gap is reduced, and when force transmitting device is lengthened, the gap becomes wider. Consequently, shortening force transmitting device 52 causes pressure to be exerted by pressure collar 20 on body part K that is surrounded by pressure collar 20.

FIG. 6 further shows that a cover 61, 74 is provided on both support device 60 and support device 70, which covers clasp deflection elements 521. In this way, covers 61, 74 serve as effective protection for the deflection elements and for the force transmitting device 52 that is wound round the deflection elements 521.

FIG. 7 illustrates a further embodiment of a blood pressure measuring device 10 according to the invention, in which the regulating device 50 is arranged on the inner side of pressure collar 20. Support device 53 consists of a flexible, roughly circular plate. A dynamic element 51 in the form of a motor and a coupling element 511 associated therewith are both mounted on the plate of support device 53. Coupling element 511 is connected to force transmitting device 52 via a transmission element 526 or transmission point 526. In the example shown, transmission point 526 is the end of the coupling element 511, configured as a tappet, via which the force transmitting device 52 is placed under tension. This force transmitting device 52 is a circular strap that encompasses pressure collar 20. In an alternative embodiment, however, (not shown in the figures), it is als possible for pressure collar 20 itself to function as the circular strap of such a blood pressure measuring device 10, as is also the case in the embodiment that will be described next, and is illustrated in FIG. 8. This makes pressure collar 20 very easy to handle. The figure shows that coupling element 511 is able to press force transmitting device outward via the transmitting element and the transmitting point 526 in direction R extending radially to the axial direction of body part K. This has the effect of reducing inner circumference I of pressure collar 20.

According to a further variant, shown in FIG. 8, regulating device 50 is also arranged on the inner side of pressure collar 20. Here too, regulating device 50 is able to apply a force to pressure collar 20 in a fiction R that extends radially to the axial direction of body part K. In this embodiment pressure collar 20 itself ahs the form of a circular strap. Here too, regulating device 50 has a support device 53 in the form of a roughly circular, flexible plate. Here too, a motor with a coupling element 511 is mounted thereon, and functions as a dynamic element 51. A cam is mounted on coupling element 511 as the force transmitting device 52, and is able to pivot in rotary direction S about the coupling element 511 of the dynamic element 51. The cam has a distal end 524 remote from the axle and a proximal 525 end close to the axle. Distal end 524 serves as a transmitting point 526 and establishes contact with pressure collar 20. In the present example, pressure collar 20 is a closed annular strap that circulates about the cam, that is to say about the force transmitting device 52 of regulating device 50. When the cam swivels about the shaft 511 of dynamic element 51, the transmitting point 526 is located by turns closer to body part K and farther away from body part K. At the same time, it guides pressure collar 20 outwards in the distant position from body part K, thereby exerting a force in the radial direction on pressure collar 20. The net effect of this is to reduce the effective inner circumference I of pressure collar 20, i.e., the inner circumference I that is able to constrict the circumference U of body part K. In a further variant (not shown), the cam may also function as a coupling element 511 and the force of dynamic element 51 may be transferred to a force transmitting device 52 which, as in the example of FIG. 7, has the form of an annular strap that encloses pressure collar 20. In a further variant (not shown) a coupling element 511 in the form of a tappet may also function as a the force transmitting device 52 at the same time, and transfer the force from the dynamic element 51 directly to pressure collar 20.

FIG. 9 shows a first example of a safety circuit designed to rapidly uncouple dynamic element 51 from the force transmitting device 52 of regulating device 50. A safety circuit of such kind enables rapid response if a fault occurs while the blood pressure measuring device is in use, such as a may be caused by a power failure or motor malfunctions. This is particularly important in order to avoid stopping the blood circulation permanently, as this might otherwise result in grave injury. With the safety circuit, it is possible to disconnect the entire force transmitting device 52 in any malfunction event, either actively by the operator, automatically by meas of the safety circuit, or by disconnecting the power supply to dynamic element 51, which immediately leads to a loosening of the pressure collar (not shown in FIG. 9).

It will be noted that hereto dynamic element 51 is a motor with a shaft 513. Shaft 513 is coupled to transmitting element 526 via a toothed gear that serves as a coupling element 511 and transfers the force of dynamic element 51 to the force transmitting device 52 (not shown) or the pressure collar 20. Transmitting element 526 and the coupling element 511 in the form of a toothes gear are pressed against the force of a spring 91 towards shaft 513 with the aid of an electromagnet 90. As soon as the power supply to the device is interrupted, the pressure exerted by the electromagnet 90 on transmitting element 526 and toothed-gear coupling element 511 is less than the force of spring 91. As a result, spring 91 now forces transmitting element 526 and toothed-gear coupling element 511 away from the shaft 513, thus breaking the contact between the toothed-gear coupling element 511 and the shaft 513. As a consequence force is no longer transmitted to transmitting element 526, and in turn no force is forwarded to the force transmitting device 52 or the pressure collar 20, so that the inner circumference of pressure collar 20 is ultimately able to expand.

FIGS. 10a, 10b and 10c show an alternative design of such a safety circuit. Hereto, the dynamic element 51 (not shown) is a motor with a shaft 513. Spring projections 93 are provided on the shaft 513. As may also be seen in the detailed view, FIG. 10c shows a view in the axial direction of shaft 513. Coupling element 511 which in this case has the form of a bobbin, may be mounted on the shaft 513. For this purpose a groove 92 is formed in coupling element 511 to accommodate spring elements 93. Spring elements 93 are each braced against a limit stop 94 on the side walls of groove 92, so that the rotation of shaft 513 may be transferred to the protruding coupling element 511. FIG. 10a shows that coupling element 511 is pressed against shaft 513 against the force of a spring 91 by means of an electromagnet 90. If the pressure exerted by electromagnetic 90 is cancelled, for example, due to a power failure, user intervention or emergency shut off, the force of spring 91 presses coupling element 511 far enough away from shaft 513 to ensure that spring elements 93 can no longer engage in groove 92 and consequently the transmission of force is interrupted.

In order to use a blood pressure collar 10 according to the invention, such as is shown in FIGS. 1, 7 and 8, pressure collar 20 is first placed around body part K. The part of the pressure collar 20 that then lays closest to artery A in this case is pressure sensor element 30. However, the tissue pressure is distributed very evenly, thereby ensuring that the pressure sensor element 30 is unaffected by position.

After the flexible pressure collar 20 has been placed over body part K, the pressure collar 20 is adjusted so that it exerts a pressure on the body part K in the pulsitile area of the patient. For this, initially a pressure is applied that is sufficient to exceed the systolic pressure prevailing in artery A. At this point, only very small pulsations, also called suprasystolic pulsations can be detected in artery A by sensor 31. Even while the pressure is elevated the pulsations are recorded and evaluated using graph analysis, in order to determine the values of the diastolic, systolic and average blood pressure, similarly to oscillonetric blood pressure measuring methods. When the systolic pressure is exceeded the inner circumference I of pressure collar 20 is slowly expanded with the aid of regulating device 50, thereby enabling blood to flow through artery A again. In this process the pressure is reduced until it is lower than the diastolic pressure. As the collar pressure is diminished, the pressure amplitude of the pulse pressure wave is measured. Pressure collar 20 is then adjusted so that it exerts a predetermined pressure on body part K, which for example, matches the pressure that was determined at the point of maximum pulsation amplitude. Then, the pulsation in artery A is recorded with the aid of sensor 31 and measuring device 32 of pressure sensor element 30 for the duration of at least one breathing cycle by the patient. This enables fluctuations in the arterial blood circulation to be measured. For this purpose, pressure collar 20 may advantageously be locked reliably at any pressure with the aid of regulating device 50, without having a damping effect on the pusitile signals of artery A. The locking mechanism may be of particularly simple design, if the dynamic element 51 in the form of a motor blocks the shaft 511 in the form of the bobbin. Such a blocking arrangement is preferably implemented by the self-inhibiting function of a reduction gear or by a detent mechanism.

Of course the invention is not limited to the exemplary embodiment described in the proceeding but can be varied or modified in many ways.

For example, it is conceivable that the force transmission device 52 may either be a rope or a V-belt. A chain device is also conceivable.

Pressure sensor element 30 may consist, for example, of a gel cushion in which a pressure sensor 31 is embedded. However, other additional sensor elements such as piezoelements, impedance electrodes or the like are also possible.

Base body 201 of pressure collar 20 may also consist of partial surfaces 21 that are polygonal, for instance triangular, octagonal, hexagonal or similar. In this case connection elements 40, even separate connecting elements 40, may be small chain links, rivets or the like. The number and shape of the deflection elements 521 is also flexible. The only important requirement is that they must be arranged and configured to ensure that the exerted force can be distributed evenly.

It will be noted the for the purpose of the present invention, it is particularly advantageous if a blood pressure measuring device 10 having a flexible pressure collar 20 that is configured to at least partially encircle a body part K and has a pressure sensor element 30, provides that the shape of pressure collar 20 is adaptable to the outer contour of body part K, and at least partly inelastic. In this case it is particularly advantageous if pressure collar 20 consists of partial surfaces 21 that are movable relative to each other, the partial surfaces preferably being flexible and inelastic.

It is also advantageous if pressure collar 20 comprises connecting elements 40, which preferably connect the movable partial surfaces together, and if pressure collar 20 comprises a regulating device 50. It is also advisable for pressure sensor element 30 to be arranged on the side of pressure collar 20 facing towards body part K.

It should also be noted that for the purposes of the present invention it is expedient if a flexible pressure collar 20 for a blood pressure measuring device 10 according to the invention provides that it is inelastic and the shape thereof is adaptable to the outer contour of a body part K. The flexible pressure collar 20 advantageously consists of partial surfaces 21 that are movable relative to each other and which are preferably of flexible and inelastic construction. It is also favorable if pressure collar 20 comprises connecting elements, which preferably connect partial surfaces 21 to each other, and if pressure collar 20 comprises a regulating device 50. In this context regulating device 50 may have at least one motor 51. It is advantageous if regulating device 50 has a force transmitting device 52, preferably a toothed belt or a cable, and if the pressure collar has a detent mechanism.

It will further be noted that for the purpose of the present invention it is advantageous if a method for non-invasive blood pressure measurement on a body part of a patient with a blood pressure measuring device 10 according to the invention comprises the steps of: a) placing the flexible pressure collar 20 on the body part K; b) adjusting the pressure collar 20 so that it exerts a pressure in the patients pulsitile range on body part K; c) recording the pulsation for a duration of at least one breathing cycle by the patient in the form of pulsation signals. In such case, it is advantageous, and possibly even preferred if the adjustment of pressure collar 20 in step b further comprises the steps i) exerting a continuously rising pressure on the body part K using pressure collar 20 until pressure collar 20 exerts a pressure in the patients pulsatile range; ii) measuring the amplitude of the pulsation; iii) increasing the pressure continuously, until the amplitude of the pulsation fall again back to a predetermined fraction of the measured maximum amplitude; iv) loosening the pressure collar 20 to a pressure value at which the maximum amplitude of the pulsation occurs; v) locking the pressure collar 20 at the pressure value set in step b). In this context it is also expedient if the pulsation signals recorded in step c) are coupled preferably with a model calculation, particularly preferably with a model calculation that contains sigmoidal transmission elements that are capable of linearization.

All features described may be essential to the invention either individually or in combination.

LIST OF REFERENCE NUMBERS

| A | Artery | U | Circumference |
|---|---|---|---|
| H | Bone | R | Direction |
| I | Inner circumference | S | Direction of |
| | | | rotation |
| K | Body part | X | Line |
| L | Longitudinal direction | Y | Line |
| 10 | Blood pressure measuring device | 433 | Fastening |
| | | 434 | Fastening |
| 20 | Pressure collar | 44 | Cutaway |
| 201 | Base body | | |
| 202 | Long side | | |
| 203 | Short side | 50 | Regulating |
| | | | device |
| 21 | Partial surface | 51 | Dynamic element |
| 211 | End area | 511 | Coupling lement |
| 22 | First end | 512 | Power cable |
| 23 | Second end | 513 | Shaft |
| | | 52 | Power |
| | | | transmitting device |
| 30 | Pressure sensor element | 521 | Deflection |
| | | | element |
| 31 | Sensor | 522 | Fastening |
| 32 | Neasuring device | 523 | Guide element |
| 33 | Power cable | 523′ | Force sensor |
| | | 524 | Axis distal end |
| 40 | Connecting element | 525 | Axis proximal |
| | | | end |
| 41 | Cutaway | 526 | Transfer |
| | | | element/Transfer |
| | | | point |
| 41′ | Transverse thread | | |
| 42 | Longitudinal thread | 526′ | Transfer |
| | | | element |
| 421 | Inner longitudinal thread | 527 | Gearwheel |
| 422 | Outer longitudinal thread | 53 | Support device |
| 43 | Joining device | | |
| 431 | Joining element | 60 | Support device |
| 432 | Deflection point | 61 | Cover |
| 70 | Support device | 90 | Electromagnet |
| 71 | Section | 91 | Spring |
| 72 | Section | 92 | Groove |
| 73 | Border | 93 | Spring |
| | | | projectction |
| 74 | Cover | 94 | Limit stop |
| 80 | Connecting area | | |

1. A blood pressure measuring device with a flexible pressure collar, configured to at least partially surround a patient's body part, and having a pressure sensor element, wherein the pressure collar is constructed as a mechanical pressure collar, particularly without fluid, preferably without a gas cushion, a gas mixture cushion and/or a fluid cushion, that the pressure collar is constructed at least partly inelastically, preferably unidirectionally inelastically, particularly preferably circumferentially inelastically, and the pressure collar comprises at least one regulating device for mechanical regulation of the inner circumference of the pressure collar when it is attached to the body part.

2. The blood pressure measuring device according to claim 1, wherein the regulating device has a least one support device, at least one dynamic element and/or at least one force transmitting element.

3. The blood pressure measuring device according to claim 2, wherein the force transmission device comprises a tensile element that is adjustable with regard to the position and/or length thereof.

4. The blood pressure measuring device according to claim 2, wherein the force transmission device comprises a cam.

5. The blood pressure measuring device according to claim 1, wherein a force can be applied to the pressure collar mechanically by the regulating device in the circumferential direction of the pressure collar.

6. The blood pressure measuring device according to claim 1, wherein a force can be applied to the pressure collar mechanically by the regulating device in the axiall direction of the body part.

7. The blood pressure measuring device according to claim 1, wherein the shape of the pressure collar is adaptable to the outer contour of the body part.

8. The blood pressure measuring device according to claim 1, wherein the regulating device of the pressure collar has a detent device.

9. The blood pressure measuring device according to claim 1, wherein the pressure collar has partial surface elements that are movable relative to each other, and wherein the partial surface elements are preferably flexible, particularly preferably flexible and at least partlay inelastic.

10. The blood pressure measuring device according to claim 1, wherein the pressure collar has connecting elements, which preferably connect the movable partial surfaces to each other.

11. The blood pressure measuring device according to claim 1, wherein the pressure sensor element is arranged on the side of the pressure collar facing towards the body part.

12. The blood pressure measuring device according to claim 1, wherein the pressure sensor element has a gel cushion, preferably a gel cushion in which a pressure sensor is embedded, and/or a gel cushion that has an element that functions according to a principle of communicating pipes.

13. A flexible pressure collar for a blood pressure measuring device according to claim 1, wherein the pressure collar is designed as a mechanical, particularly a fluid-free, preferably a gas cushion, gas mixture cushion and/or fluid cushion free pressure collar, that the pressure collar is at least partly inelastic, preferably unidirectionally inelastic, particularly preferably circumferentially inelastic, and the pressure collar has at least one regulating device for mechanical regulation of the inner circumference of the pressure collar when applied to the body part.

14. The flexible pressure collar according to claim 13, wherein the collar has partial surfaces that are movable relative to each other.

15. A method for non-invasive blood pressure measurement on a body part of a patient, with a blood pressure measuring device according to claim 1, comprising:
placing the flexible pressure collar on the body part;
adjusting the pressure collar that exerts a pressure in the pulsatile range of the patient on the body part; and
recording the pulsation for the duration of at least one breathing cycle for the patient in the form or pulsation signals.

16. The method according to claim 15, wherein the adjustment of the pressure collar in the adjusting the pressure collar comprising:
exerting a continuously rising pressure on the body part via the pressure collar, until the pressure collar exerts a pressure in the pulsatile range of the patient;
measuring the amplitude of the pulsations;
continuously increasing the pressure until the amplitudes of the pulsations fall back to a predetermined fraction of the measured maximum amplitude;
loosening the pressure collar to reach a pressure value in the pulsatile range; and
locking the pressure collar at the pressure value set in the loosening the pressure collar.

17. The method according to claim 15, wherein the pulsation signals recorded in the recording the pulsation, may be linearized preferably with the aid of a model calculation, particularly preferably with a model calculation containing sigmoidal transfer elements.
